(19) United States
(12) Patent Application Publication
(10) Pub. No.: US 2025/0366751 A1
(43) Pub. Date: Dec. 4, 2025

(54) SYSTEMS AND METHODS FOR IDENTIFYING PHYSIOLOGICAL STATES OF HUMAN SUBJECTS

(71) Applicant: Toyota Motor Engineering & Manufacturing North America, Inc., Plano, TX (US)

(72) Inventors: Hossein Hamidi Shishavan, Westland, MI (US); Ercan Mehmet Dede, Ann Arbor, MI (US); Paul Donald Schmalenberg, Ann Arbor, MI (US)

(21) Appl. No.: 18/732,962

(22) Filed: Jun. 4, 2024

(51) Int. Cl. A61B 5/18; A61B 5/00; G16H 10/20

(52) U.S. Cl. CPC A61B 5/18; A61B 5/6802; A61B 5/74; G16H 10/20

(57) ABSTRACT

Systems and methods for identifying physiological states of human subjects are disclosed herein. In one embodiment, a system receives, from one or more sensors, physiological signals of a human subject. The system processes the physiological signals to extract feature signals as time series. The system identifies change points in the feature signals. The system identifies critical change points in the feature signals by applying a voting process to the change points in the feature signals. The system partitions the feature signals into segments based on the critical change points. The system detects a predetermined physiological state (an anomalous physiological state) of the human subject by applying clustering and probabilistic analysis to the segments. In response to detecting the predetermined physiological state, the system automatically takes an action to assist the human subject.

### TECHNICAL FIELD

The subject matter described herein generally relates to the analysis of time series data and, more specifically, to systems and methods for identifying physiological states of human subjects.

### BACKGROUND

In a variety of applications, times series are analyzed to identify abrupt changes in the signals. These abrupt changes are sometimes referred to in the art as “anomalies.” For example, in some applications, physiological signals from a human subject are analyzed to identify anomalies such as emotions, stress, and drowsiness. Identifying such anomalies reliably is a challenging problem. Conventional systems fail to account for the differences among human subjects and for the variations in an individual subject's baseline or rest state that occur within a single day and that evolve over longer periods. Also, conventional approaches to anomaly detection using deep learning algorithms require a tremendous amount of data to train.

### SUMMARY

An example of a system for identifying physiological states of human subjects is presented herein. In one embodiment, the system comprises a processor and a memory storing machine-readable instructions that, when executed by the processor, cause the processor to receive, from one or more sensors, physiological signals of a human subject. The memory also stores machine-readable instructions that, when executed by the processor, cause the processor to process the physiological signals to extract feature signals as time series. The memory also stores machine-readable instructions that, when executed by the processor, cause the processor to identify change points in the feature signals. The memory also stores machine-readable instructions that, when executed by the processor, cause the processor to identify critical change points in the feature signals by applying a voting process to the change points in the feature signals. The memory also stores machine-readable instructions that, when executed by the processor, cause the processor to partition the feature signals into segments based on the critical change points. The memory also stores machine-readable instructions that, when executed by the processor, cause the processor to detect a predetermined physiological state of the human subject by applying clustering and probabilistic analysis to the segments. The memory also stores machine-readable instructions that, when executed by the processor, cause the processor to take automatically, in response to detecting the predetermined physiological state, an action to assist the human subject.

Another embodiment is a non-transitory computer-readable medium for identifying physiological states of human subjects and storing instructions that, when executed by a processor, cause the processor to receive, from one or more sensors, physiological signals of a human subject. The instructions also cause the processor to process the physiological signals to extract feature signals as time series. The instructions also cause the processor to identify change points in the feature signals. The instructions also cause the processor to identify critical change points in the feature signals by applying a voting process to the change points in the feature signals. The instructions also cause the processor to partition the feature signals into segments based on the critical change points. The instructions also cause the processor to detect a predetermined physiological state of the human subject by applying clustering and probabilistic analysis to the segments. The instructions also cause the processor to take automatically, in response to detecting the predetermined physiological state, an action to assist the human subject.

Another embodiment is a method of identifying physiological states of human subjects. The method includes receiving, from one or more sensors, physiological signals of a human subject. The method also includes processing the physiological signals to extract feature signals as time series. The method also includes identifying change points in the feature signals. The method also includes identifying critical change points in the feature signals by applying a voting process to the change points in the feature signals. The method also includes partitioning the feature signals into segments based on the critical change points. The method also includes detecting a predetermined physiological state of the human subject by applying clustering and probabilistic analysis to the segments. The method also includes taking automatically, in response to detecting the predetermined physiological state, an action to assist the human subject.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

FIG. 1 illustrates one embodiment of a vehicle within which systems and methods disclosed herein may be implemented.

FIG. 2 is a block diagram of a physiological-state identification system, in accordance with an illustrative embodiment of the invention.

FIG. 3 illustrates identifying change points and anomalous segments in a feature signal, in accordance with an illustrative embodiment of the invention.

FIG. 4 illustrates identifying critical change points across a set of feature signals using a voting process, in accordance with an illustrative embodiment of the invention.

FIG. 5 illustrates an electronic self-report questionnaire for the driver of a vehicle, in accordance with an illustrative embodiment of the invention.

FIG. 6 is a flowchart of a method of identifying physiological states of human subjects, in accordance with an illustrative embodiment of the invention.

To facilitate understanding, identical reference numerals have been used, wherever possible, to designate identical elements that are common to the figures. Additionally, elements of one or more embodiments may be advantageously adapted for utilization in other embodiments described herein.

### DETAILED DESCRIPTION

Various embodiments of systems and methods for identifying physiological states of human subjects described herein address the shortcomings of the prior art in that they account for individual differences among human subjects, and they do not require years' worth of data to train, as some conventional deep-learning-based solutions do. For example, using the techniques described herein, even a single detected instance of an anomaly for a given individual human subject (hereinafter sometimes referred to as simply a “subject” or a “human”) can be sufficient to support future identification of a similar anomaly in that subject. In the various embodiments described herein, an unsupervised machine-learning approach to time-series anomaly detection is applied to the problem of identifying specific predetermined physiological states in subjects. In some embodiments, the disclosed techniques are applied to improving the safety of a driver in a vehicle. In other embodiments, the disclosed techniques can be used to assist subjects in a variety of other settings.

As mentioned above, one of the most challenging aspects of the kind of time-series analysis discussed herein is identifying the anomalies in the first instance. Herein, an “anomaly” refers to a sudden change in one or more of a subject's physiological signals in connection with a medical condition of the subject, a response to a stimulus in the environment (e.g., one or more emotions), or a behavior of the subject (e.g., a driver of a vehicle suddenly applying the brakes or swerving). In embodiments, a physiological-state identification system receives, from one or more sensors, one or more physiological signals of a human subject. The system extracts features from the one or more physiological signals. That is, the system extracts, from the one or more physiological signals, feature signals in the form of time series. The system then identifies change points in the feature signals. In some embodiments, the system does so using a change point detection (CPD) algorithm. To identify anomalies for a particular individual subject more accurately and reliably than conventional approaches, the system identifies critical change points in the feature signals by applying a voting process to the change points across the analyzed feature signals. Based on the identified critical change points, the system partitions (divides) the feature signals into segments. That is, the critical change points delimit distinct segments in the feature signals that can be further analyzed to detect anomalies in particular segments.

Once the feature signals have been segmented, the system applies clustering and probabilistic analysis to the segments to identify anomalous segments. Through the performance-evaluation process explained below, the anomalous segments can be mapped to specific predetermined physiological states. Examples of such predetermined physiological states include, without limitation, a particular kind of symptomatic illness (e.g., a heart condition or motion sickness), an emotion (or a combination of emotions), stress, intoxication, sleepiness, fatigue, and drowsiness.

Once the system has detected a particular predetermined physiological state (i.e., an anomalous physiological state) of the subject, the system, in response, automatically takes an action to assist the subject with regard to the detected predetermined (anomalous) physiological state. Examples of such assistive actions are discussed further below.

Another important aspect of the various embodiments described herein is evaluating the performance of the unsupervised machine-learning process summarized above and adjusting the parameters of the algorithm accordingly. This involves comparing labeled anomalies (detected physiological states) with ground-truth data. For example, in one embodiment, the system compares a labeled physiological state with (1) a self-report questionnaire answered by the subject, (2) event timestamps captured by one or more environmental sensors in the environment of the subject, or (3) both the questionnaire data and the event-timestamp data. Further, in some embodiments, medical records can be used as ground-truth data for existing medical disorders or illnesses. Based on that comparison, the system can adjust one or more thresholds of the CPD algorithm to improve the accuracy of future anomaly detection and labeling (identification). For example, when a similar anomaly is detected in the future, the system can identify the anomaly as being associated with a particular physiological/emotional state based on the previously learned mapping between that kind of anomaly and a specific physiological state of that individual subject. Moreover, in some embodiments, the system also tracks both short-term and long-term changes in a subject's baseline (rest) state to improve the accuracy of anomaly detection and associated physiological-state identification.

As mentioned above, in some embodiments a physiological-state identification system is installed in a vehicle to protect the safety of the driver and other vehicle occupants. In other embodiments, a physiological-state identification system is embodied in a mobile device that is carried or worn by the human subject in settings that may not involve a vehicle. For example, detecting a person's emotional state in the workplace (e.g., to prevent violent incidents among co-workers) is a topic of ongoing active research. Vehicular embodiments are described in detail below for purposes of explanation and illustration, but the principles and techniques of the invention are not limited to that particular kind of embodiment. Instead, those principles and techniques can be applied to a variety of different applications involving the identification of physiological states in human subjects.

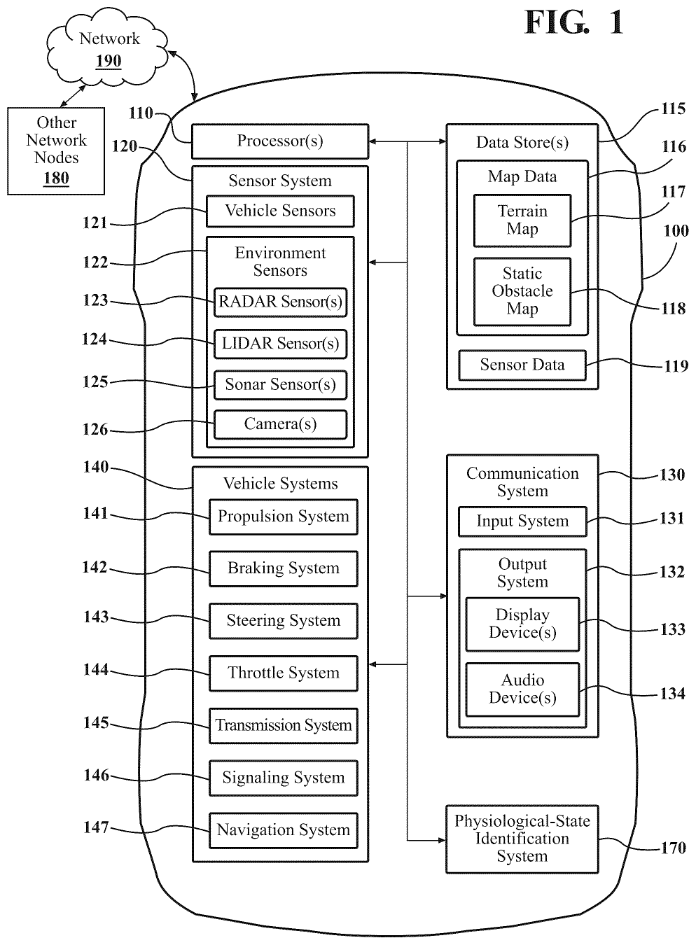

Referring to FIG. 1, an example of a vehicle 100, in which systems and methods disclosed herein can be implemented, is illustrated. The vehicle 100 can include a physiological-state identification system 170 (hereinafter sometimes referred to as simply the “system 170”) or components and/or modules thereof. As used herein, a “vehicle” is any form of motorized land transport. For example, in some embodiments, the vehicle 100 is an automobile. Herein, a vehicle 100 is sometimes referred to as an “ego vehicle.” An “ego vehicle” is a vehicle 100 from whose point of view an onboard physiological-state identification system 170 operates to assist the driver of the vehicle 100. That is, in the various scenarios discussed herein, a vehicle in which a physiological-state identification system 170 has been installed is considered to be an “ego vehicle.”

The vehicle 100 also includes various elements. It will be understood that, in various implementations, it may not be necessary for the vehicle 100 to have all the elements shown in FIG. 1. The vehicle 100 can have any combination of the various elements shown in FIG. 1. Further, the vehicle 100 can have additional elements to those shown in FIG. 1. In some arrangements, the vehicle 100 may be implemented without one or more of the elements shown in FIG. 1, including the system 170. While the various elements are shown as being located within the vehicle 100 in FIG. 1, it will be understood that one or more of these elements can be located external to the vehicle 100 or be part of a system that is separate from vehicle 100. Further, the elements shown may be physically separated by large distances.

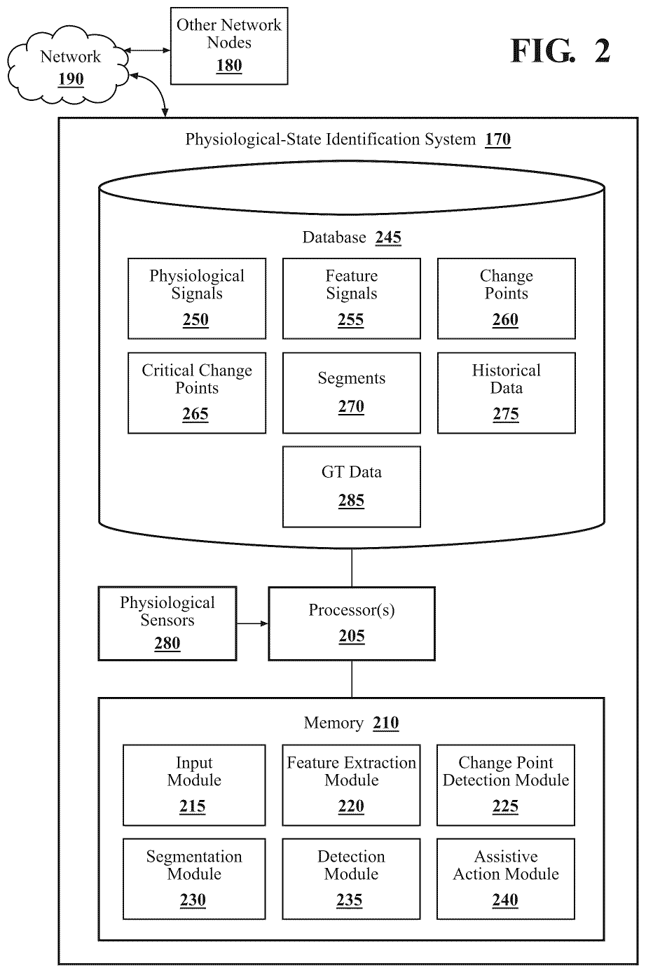

Some of the possible elements of the vehicle 100 are shown in FIG. 1 and will be mentioned in connection with subsequent figures. However, a description of many of the elements in FIG. 1 will be provided after the discussion of FIGS. 2-6 for purposes of brevity of this description. Additionally, it will be appreciated that for simplicity and clarity of illustration, where appropriate, reference numerals have been repeated among the different figures to indicate corresponding or analogous elements. In addition, the discussion outlines numerous specific details to provide a thorough understanding of the embodiments described herein. Those skilled in the art, however, will understand that the embodiments described herein may be practiced using various combinations of these elements.

Sensor system 120 can include one or more vehicle sensors 121. Vehicle sensors 121 can include one or more positioning systems such as a dead-reckoning system or a global navigation satellite system (GNSS) such as a global positioning system (GPS). Vehicle sensors 121 can also include Controller-Area-Network (CAN) sensors (sometimes herein referred to as “CAN-bus sensors”) that output, for example, speed and steering-angle data pertaining to vehicle 100. Sensor system 120 can also include one or more environment sensors 122. Environment sensors 122 generally include, without limitation, radar sensor(s) 123, Light Detection and Ranging (LIDAR) sensor(s) 124, sonar sensor(s) 125, and camera(s) 126.

Communication system 130 includes an input system 131 and an output system 132. The output system 132 can include components such as one or more displays 133 and one or more audio devices 134. As explained further below, the system 170 can use display device(s) 133, audio device(s) 134, and/or other kinds of sensors, including a brain-machine interface (BMI), to present a self-report questionnaire (a source of ground-truth data used in evaluating the performance of the unsupervised machine-learning algorithm and its variations described herein) to the driver of vehicle 100 and to collect responses to the questionnaire from the driver. As those skilled in the art are aware, display device(s) 133 and audio device(s) 134 can be used, in general, to communicate with the driver or other vehicle occupants.

As shown in FIG. 1, vehicle 100 may, in some embodiments, communicate with one or more other network nodes (servers, edge servers, infrastructure devices, other connected vehicles, etc.) 180 via a network 190. In FIG. 1, network 190 represents any of a variety of wired and wireless networks. For example, in communicating directly with another vehicle, sometimes referred to as vehicle-to-vehicle (V2V) communication, vehicle 100 can employ a technology such as dedicated short-range communication (DSRC) or Bluetooth Low Energy (BLE). In communicating with a cloud or edge server or a roadside unit (RSU), vehicle 100 can use a technology such as cellular data (LTE, 5G, 6G, etc.). In some embodiments, network 190 includes the Internet.

Referring to FIG. 2, it illustrates one embodiment of a physiological-state identification system 170. The system 170 includes one or more processors 205. In some embodiments, the one or more processors 205 coincide, partially or fully, with the one or more processors 110 of vehicle 100. In such an embodiment, the system 170 may access one or more of the one or more processors 110 through a data bus or another communication path. In other embodiments, the one or more processors 205 are separate from the one or more processors 110. As shown in FIG. 2, the system 170 includes a memory 210 that stores an input module 215, a feature extraction module 220, a change point detection module 225, a segmentation module 230, a detection module 235, and an assistive action module 240. The memory 210 is a random-access memory (RAM), read-only memory (ROM), a hard-disk drive, a flash memory, or other suitable memory for storing the modules 215, 220, 225, 230, 235, and 240. The modules 215, 220, 225, 230, 235, and 240 are, for example, computer-readable instructions that, when executed by the one or more processors 205, cause the one or more processors 205 to perform the various functions disclosed herein.

In the embodiment of FIG. 2, the system 170 includes one or more physiological sensors 280 that produce physiological signals 250 of a human subject (e.g., the driver of a vehicle 100). The physiological sensors 280 are embedded in the environment of the subject (e.g., in the driver seat of a vehicle 100). Depending on the type of a given sensor, the physiological sensors 280 may or may not need to be in physical contact with the subject. In some embodiments, the physiological sensors 280 are separate from the system 170, and the system 170 communicates with the one or more physiological sensors 280.

As shown in FIG. 2, the system 170, in some embodiments, can communicate with one or more other network nodes (servers, edge servers, infrastructure devices, other connected vehicles) 180 via a network 190, as discussed above. Though not shown in FIG. 2, the system 170, in some embodiments, interfaces with the communication system 130 of vehicle 100, particularly display device(s) 133 and audio device(s) 134, as discussed above.

The system 170 can store various kinds of data in a database 245. In the embodiment of FIG. 2, system 170 stores physiological signals 250, feature signals 255, change points 260, critical change points 265, segments 270, historical data 275, and ground-truth (GT) data 285. GT data 285 includes, for example, self-report questionnaire data from the subject and event-timestamp data captured by one or more environmental sensors in the environment of the subject, such as the environment sensors 122 of vehicle 100 (refer to FIG. 1). Database 245 can also store morphological data regarding the subject, such as age, gender, etc. These various types of data are discussed further below.

Before discussing the functions performed by the modules 215, 220, 225, 230, 235, and 240, a brief overview of the processing operations the system 170 performs will first be provided. The high-level steps are as follows: (1) Capture physiological signals 250 from the physiological sensors 280; (2) Clean and de-noise the physiological signals 250; (3) Extract features from the physiological signals 250 (i.e., extract feature signals 255 in in the form of time series); (4) Select the particular features (feature signals 255) that are most relevant to the analysis of a given hypothesized anomaly; (5) Detect change points 260 in each feature signal 255 using, e.g., a CPD algorithm; (6) Identify the critical change points (CCPs) 265 in the feature signals 255 using a voting process; (7) Segment the feature signals 255 based on the CCPs 265; (8) Apply clustering and probabilistic analysis to the segments 270 to identify and label the anomalous segments. Additionally, as discussed above, the system 170 can evaluate the performance of the foregoing unsupervised machine-learning algorithm by comparing a labeled physiological state (e.g., a particular emotion such as “annoyance”) with (1) a self-report questionnaire answered by the human subject, (2) event timestamps captured by one or more environmental sensors in the environment of the subject, or (3) both the questionnaire data and the event-timestamp data. As discussed above, in some embodiments, medical records can also be used as ground-truth data for existing medical disorders or illnesses. Based on that comparison, the system 170 can adjust one or more thresholds of the CPD algorithm to improve the accuracy of future anomaly detection and labeling.

Input module 215 generally includes machine-readable instructions that, when executed by the one or more processors 205, cause the one or more processors 205 to receive, from the one or more physiological sensors 280, one or more physiological signals 250 of a human subject. Some examples of physiological signals 250 include, without limitation, electrocardiogram (ECG), electroencephalogram (EEG), photoplethysmography (PPG), electrodermal activity (EDA) (also known as “skin conductance”), temperature, and signals related to respiration.

In some embodiments, input module 215 also includes machine-readable instructions that, when executed by the one or more processors 205, cause the one or more processors 205 to clean and de-noise the physiological signals 250. This can include removing noisy segments and/or reconstructing lost data. The cleaning and de-noising process can account for effects such as baseline wandering, muscle artifacts, powerline interference, and subject-electrode motion artifacts. For example, baseline wandering can be mitigated by using respiratory patterns captured by an ECG or respiration sensor. As a further example, noise due to subject-electrode motion artifacts can be mitigated using an adaptive filter that processes electrode motion measured by an accelerometer.

Feature extraction module 220 generally includes machine-readable instructions that, when executed by the one or more processors 205, cause the one or more processors 205 to process the one or more physiological signals 250 to extract feature signals 255 as time series. This process may be termed “feature extraction.” The feature signals 255 represent various statistical, time-domain, and frequency-domain characteristics of the raw physiological signals 250. Examples of feature signals 255 include, without limitation, mean heart rate (HR) derived from an ECG, heart-rate variability (HRV), ECG-Derived Respiration (EDR), Pulse Transit Time (PTT) associated with continuous blood pressure, the root-mean-square of successive differences between normal heartbeats (RMSSD), low-frequency-to-high-frequency ratio of HRV (LF/HF ratio), inter-beat interval of an ECG or PPG signal (IBIS), and PPG entropy. The feature signals 255 can also include the mean or variance of a raw physiological signal 250 as a function of time or a time series that identifies the type of statistical distribution (e.g., uniform, Gaussian, Gamma) exhibited by a given raw physiological signal 250 as a function of time.

In some embodiments, feature extraction module 220 also includes machine-readable instructions that, when executed by the one or more processors 205, cause the one or more processors 205 to select, from among the available feature signals 255, a subset of most highly relevant feature signals 255 for the analysis to be performed. For example, if the system hypothesizes a particular physiological state such as an emotion (e.g., “irritation”), the feature signals 255 most pertinent to detecting that physiological state can be selected. This process of feature selection can include the use of techniques such as Correlation-Based Feature Selection (CFS), Consistency-Based Filters, and Lasso regularization. Lasso regularization, for example, helps to narrow down the set of most-relevant features for detecting a particular type of anomaly in a time series.

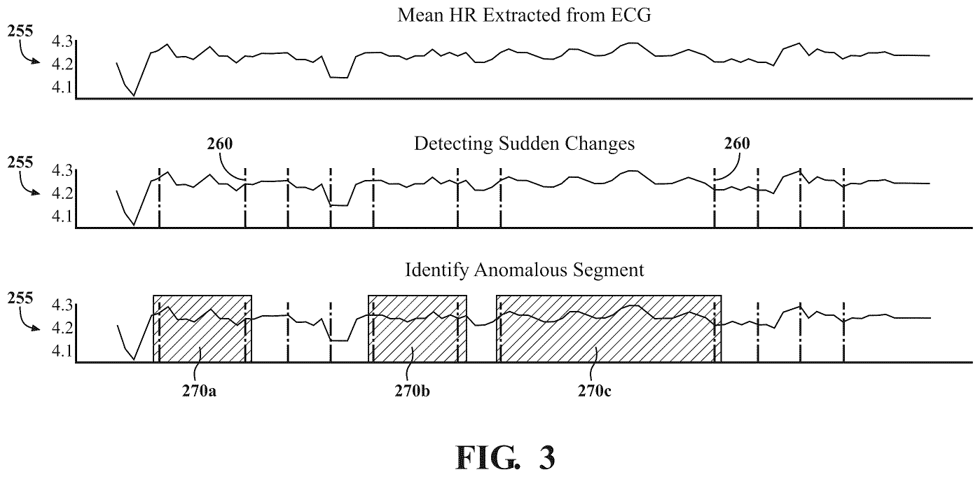

Change point detection module 225 generally includes machine-readable instructions that, when executed by the one or more processors 205, cause the one or more processors 205 to identify change points in the feature signals 255. As explained above, in some embodiments, change point detection module accomplishes this using a CPD algorithm. As those skilled in the art are aware, a CPD algorithm detects abrupt changes in the statistical distribution of a time series. An abrupt change can, for example, occur in the mean, variance, correlation length, or type of distribution (probability density function) itself. As those skilled in the art are aware, the analysis performed by a CPD algorithm is deterministic, but, in the embodiments disclosed herein, there is also a learned component, particularly in the algorithm's anomaly-detection thresholds (e.g., the amount by which the mean or variance of a physiological signal 250 must change to be considered an anomaly). As discussed above, the anomaly-detection thresholds can be adjusted over time based on a comparison of labeled anomalies (detected physiological states) with GT data 285 to improve the detection of specific physiological states (target states). A simple example of analyzing a feature signal 255 for change points 260 is illustrated in FIG. 3.

FIG. 3 illustrates identifying change points 260 and anomalous segments in a feature signal 255, in accordance with an illustrative embodiment of the invention. In the simplified example of FIG. 3, three vertically stacked copies of a plot of a single feature signal 255, mean HR extracted from an ECG, are shown for purposes of annotation and explanation. The independent variable in FIG. 3 is time (i.e., the mean HR signal is a time series). As shown in FIG. 3, a CPD algorithm identifies change points 260 (abrupt changes, in accordance with a predetermined threshold) in the time series. Only two of the change points have been labeled with a reference numeral in FIG. 3 for clarity. The bottom plot illustrates that the change points can act as delimiters for partitioning the mean HR signal into segments 270 between change points 260. As discussed further below, one or more clustering algorithms can be applied to the segments 270, and the individual segments 270 can be assigned a probability of anomaly. Three examples of segments are identified in FIG. 3, segment 270a, segment 270b, and segment 270c. Such segments 270 can be compared to identify anomalous segments, which tend to be the most dissimilar compared with other segments. Such anomalous segments can correspond, e.g., to a symptomatic illness (e.g., a heart condition or motion sickness), an emotion, stress, drowsiness, etc., as discussed above.

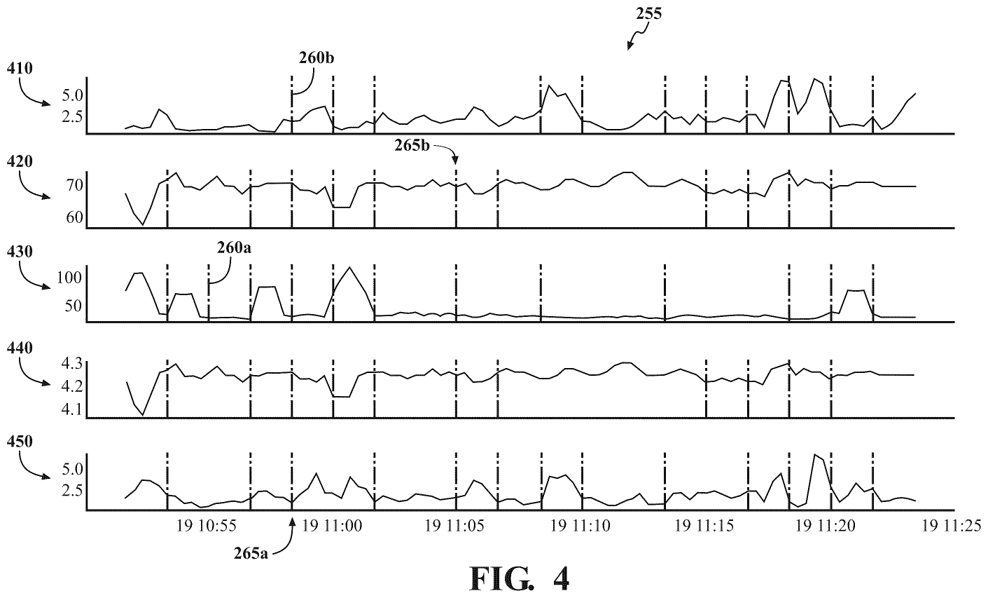

Change point detection module 225 also includes machine-readable instructions that, when executed by the one or more processors 205, cause the one or more processors 205 to identify critical change points (CCPs) in the feature signals 255 by applying a voting process to the change points 260 in the feature signals 255. This is illustrated in FIG. 4.

FIG. 4 illustrates identifying critical change points 265 across a set of feature signals 255 using a voting process, in accordance with an illustrative embodiment of the invention. The example of FIG. 4 includes a set of five feature signals 255: LF/HF ratio 410, IBIS 420, RMSSD 430, mean HR 440, and PPG Entropy 450. Using a CPD algorithm, change point detection module 225 has identified, in each of the time series, a number of change points 260 whose corresponding time instants are marked in FIG. 4 with dotted vertical lines. Note that change point 260a is the only change point that occurs at that particular time instant across the five feature signals 255. Such a point in time is not deemed a critical change point 265 and is, consequently, ignored. In contrast, all five feature signals 255 have a change point 260 at the time instant corresponding to change point 260b. In some embodiments, change point detection module 225 identifies, as a critical change point 265, a point in time at which all the selected feature signals 255 under analysis have a change point 260 (unanimous voting). An example of such a unanimous critical change point 265 is critical change point 265a in FIG. 4. In other embodiments, a majority voting system is employed. For example, in such an embodiment the time instant corresponding to critical change point 265b has change points 260 in four of the five feature signals 255-a majority of the feature signals 255. Not all change points 260 and critical change points 265 are labeled with reference numerals in FIG. 4 for the sake of clarity. It should be noted that, in some embodiments, considerably more than five feature signals 255 are involved in the analysis and voting process. For example, in one embodiment, 20 different feature signals 255 are analyzed, and the voting occurs across the 20 feature signals 255.

Segmentation module 230 generally includes machine-readable instructions that, when executed by the one or more processors 205, cause the one or more processors 205 to partition the feature signals 255 into segments 270 based on the critical change points 265 identified by change point detection module 225. As discussed above, the critical change points 265 serve as delimiters for dividing the feature signals 255 into segments 270, as illustrated for the simple case of a single feature signal 255 (mean HR) in FIG. 3. In other words, the segments 270 are the portions of the feature signals 255 between critical change points 265.

Detection module 235 generally includes machine-readable instructions that, when executed by the one or more processors 205, cause the one or more processors 205 to detect a predetermined physiological state of the human subject by applying clustering and probabilistic analysis to the segments. As discussed above, a probability-of-anomaly can be assigned to each segment 270 designated by segmentation module 230. In clustering the segments, detection module 235 employs density, distribution, or distance-based algorithms such as K-means clustering or a Gaussian Mixture Model (GMM) to label the segments 270 (e.g., to label them as anomalous). This clustering and assignment of probability-of-anomaly ultimately identifies the segment 270 in which a hypothesized physiological state of interest (“target state”) most likely occurred. Importantly, this clustering process is performed (repeated) for each target state. For example, the process might be performed for a symptomatic medical condition such as “motion sickness” and again for “irritation” and again for “stress.” The comparisons among segments 270 can also involve the individual subject's past physiological data (historical data 275 in FIG. 2). In comparing segments 270 to determine their similarities and differences, detection module 235 can employ techniques such as dynamic time warping (DTW). This is particularly useful for comparing segments 270 of different durations.

As discussed above, examples of the predetermined physiological states that detection module 235 detects include, without limitation, a particular kind of symptomatic illness (e.g., a heart condition or motion sickness), an emotion (or a combination of emotions), stress, and drowsiness.

Assistive action module 240 generally includes machine-readable instructions that, when executed by the one or more processors 205, cause the one or more processors 205 to take automatically, in response to detecting the predetermined physiological state, an action to assist the human subject with regard to the detected predetermined physiological state. The action taken can vary depending on the embodiment of physiological-state identification system 170 and the nature of the particular detected physiological state. In an embodiment in which physiological-state identification system 170 is installed in a vehicle 100, the action can, without limitation, include one or more of the following: (1) notifying the subject regarding the detected predetermined physiological state; (2) notifying the subject regarding a potentially unsafe driving condition stemming from the detected predetermined physiological state; (3) notifying the subject regarding a symptomatic illness (e.g., a heart condition or motion sickness) of the subject based on the detected predetermined physiological state; (4) advising the subject regarding mitigation of the detected physiological state (e.g., recommending that the subject take a break from driving or offering to turn on a particular kind of music in the vehicle to calm the driver or to increase the driver's level of alertness); (5) suggesting to the subject that the human subject take an alternate route to a planned destination (e.g., a route that is less stressful for the driver); (6) communicating with a person associated with the subject (e.g., a friend or family member) or an artificial-intelligence (AI) system to obtain help for the subject; (7) assuming at least partial control over operation of the vehicle (route-planning, steering, braking, and/or acceleration) to mitigate a potentially unsafe driving condition stemming from the detected physiological state, where the vehicle 100 includes an automated driving system or an Advanced Driver-Assistance System (ADAS); and (8) alerting other drivers on the roadway to potential danger stemming from the detected predetermined physiological state of the subject.

In some embodiments, physiological-state identification system 170 is embodied in a mobile device that is worn or carried by the subject instead of being installed in a vehicle. In such an embodiment, the physiological sensors 280 may be either integral with the mobile device (e.g., a smart watch or smart phone) or separate from the mobile device. In this kind of non-vehicular mobile embodiment, the automatic action assistive action module 240 takes can include, without limitation, one or more of the following: (1) notifying the subject regarding the detected predetermined physiological state; (2) notifying the subject regarding a symptomatic illness of the subject (e.g., a heart condition) based on the detected physiological state; (3) advising the subject regarding mitigation of the detected physiological state (e.g., suggesting that the subject take a break or engage in a relaxing activity, suggesting that the subject seek medical attention, etc.); and (4) communicating with a person associated with the subject (e.g., a friend or family member) or an AI assistant to obtain help for the subject.

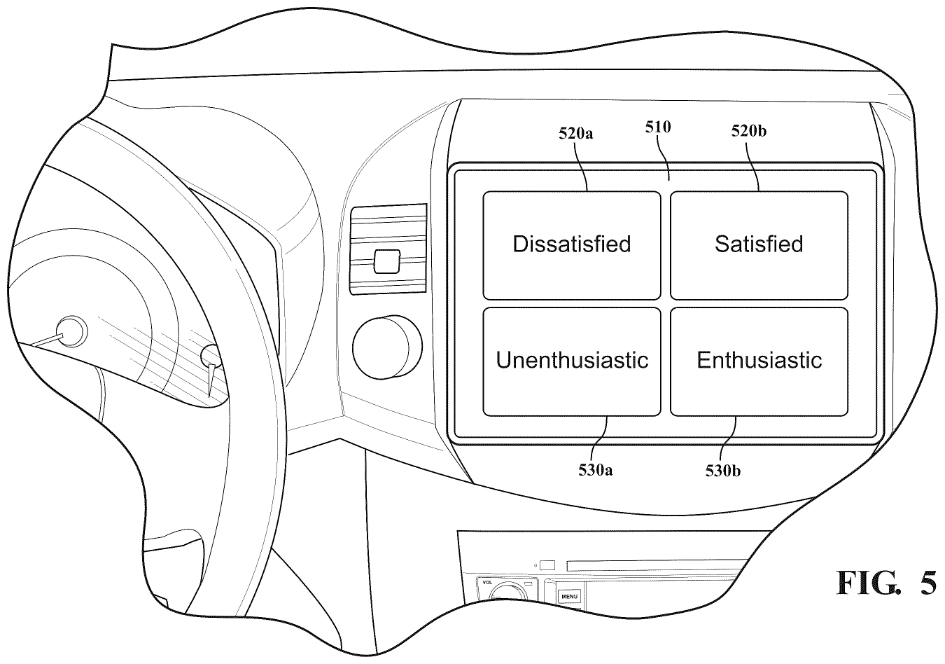

As discussed above, the system 170 can generate GT data 285 with which labeled anomalies (detected physiological states) are compared to build, over time, the capability in the system 170 to identify specific physiological states. For example, FIG. 5 illustrates an electronic self-report questionnaire 510 for the driver of a vehicle 100, in accordance with an illustrative embodiment of the invention. As events and environmental stimuli occur in real time as the driver operates the vehicle, the questionnaire 510 can present questions about those events or stimuli to which the driver responds. This provides system 170 with ground-truth data concerning the driver's responses to the time-correlated events and environmental stimuli. In the example of FIG. 5, the questionnaire 510 presents pairs of opposite responses from which the driver chooses. In this particular example, the driver, in response to a situation/event/stimulus, can choose between response 520a (“Dissatisfied”) and response 520b (“Satisfied”). Similarly, the driver can choose between response 530a (“Unenthusiastic”) and response 530b (“Enthusiastic”). The subject's responses are recorded and timestamped for later correlation with feature signals 255. In general, the responses provided to the subject via questionnaire 510 reflect the target physiological state of interest. For example, for motion sickness, the driver might be asked to choose between the responses “Feel nauseous” and “Don't feel nauseous.”

For the identification of specific emotions, tools such as Russel's Circumplex Model for emotions (1980) can be used as a guide in formulating responses for the questionnaire 510. Such a model accounts for different degrees of arousal/activation on a vertical axis and different degrees of pleasantness/unpleasantness (valence) on the horizontal axis. Though the example in FIG. 5 shows only one icon layer, in other embodiments the questionnaire 510 is presented to the subject in multiple icon layers to support more deeply identifying a particular emotional state in Russel's Circumplex Model.

As discussed above, in connection with a questionnaire 510, the system 170 can use display device(s) 133, audio device(s) 134, and/or other sensor systems, including a BMI. For example, in one embodiment, the BMI is a closed-loop steady-state visual evoked potentials (SSVEP) system that enables the driver to respond to a questionnaire 510 without having to physically touch user-interface elements such as a touchscreen. Such as system synchronizes visual stimuli, processes EEG signals, and selects target icons (e.g., questionnaire responses) in real time.

Moreover, other methods of collecting responses to the questionnaire 510 are used in some embodiments. For example, in some embodiments, the system 170 uses speech recognition or a gesture-based user interface to collect the driver's responses to the questionnaire 510.

Additionally, GT data 285 can be gathered from one or more environment sensors 122 of a vehicle 100. Such data can be timestamped to record when detected events, conditions, and stimuli in the environment of the subject occurred. For example, the system 170 may detect that the driver has braked or swerved suddenly (indicating that something important is happening). Another example is the system 170 detecting (e.g., via an interior camera in vehicle 100) a shocked or surprised expression on the driver's face in response to a stimulus in the environment. Yet another example is detecting that an airplane is passing overhead at low altitude, making a lot of noise. Another example is detecting that another road user (e.g., another vehicle) has suddenly swerved into the path of the ego vehicle 100 or that another motorist has honked his or her vehicle's horn nearby, startling or irritating the driver of the ego vehicle 100). Many more examples could be mentioned, including, without limitation, detecting loud construction equipment (e.g., a jackhammer) nearby or detecting that the driver of the ego vehicle 100 swerved suddenly to avoid an obstacle in the roadway. The central concept in these various examples is detecting an event or condition in the environment of the driver that could produce a physiological response (emotion, stress, etc.) and timestamping the identified event or condition.

One advantage of the embodiments described herein is that they help to resolve the time lag between two signals (e.g., between GT data 285 indicating a sudden-braking event and the driver's physiological response to that stimulus, as reflected in the physiological signals 250 and the feature signals 255 derived therefrom). The techniques described above can be used to effectively remove the time lag between the two signals.

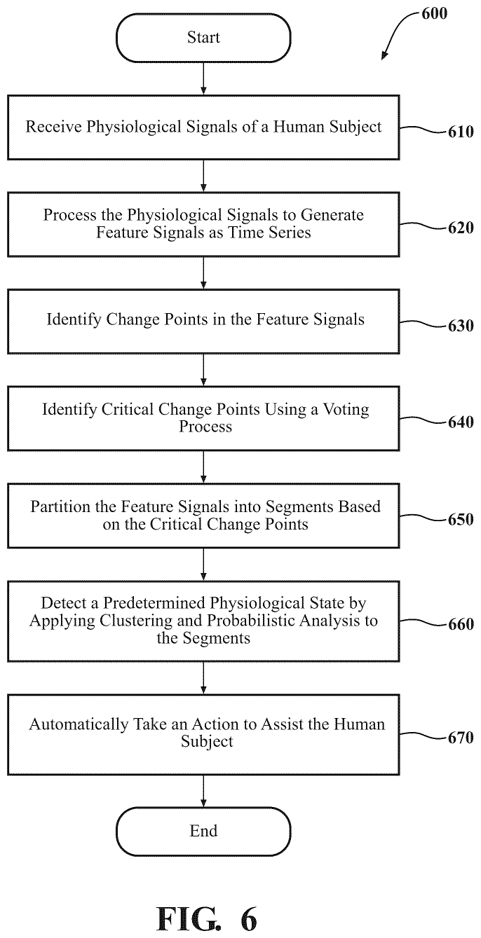

FIG. 6 is a flowchart of a method of identifying physiological states of human subjects, in accordance with an illustrative embodiment of the invention. Method 600 will be discussed from the perspective of the system 170 shown in FIG. 2. While method 600 is discussed in combination with the system 170, it should be appreciated that method 600 is not limited to being implemented within the system 170, but the system 170 is instead one example of a system that may implement method 600. As discussed above, in some embodiments a system similar to system 170 is implemented in a mobile device that is worn or carried by the human subject instead of being installed in a vehicle 100. Though the settings are different, the techniques and principles disclosed herein are equally applicable.

At block 610, input module 215 receives, from one or more physiological sensors 280, one or more physiological signals 250 of a human subject. As discussed above, some examples of physiological signals 250 include, without limitation, ECG, EEG, PPG, EDA, temperature, and respiration-related signals. As also discussed above, input module 215, in some embodiments, cleans and de-noises the physiological signals 250.

At block 620, feature extraction module 220 processes the one or more physiological signals 250 to extract feature signals 255 as time series. As discussed above, this process may be termed “feature extraction.” The feature signals 255 represent various statistical, time-domain, and frequency-domain characteristics of the raw physiological signals 250. Examples of feature signals 255 include, without limitation, mean HR, HRV, EDR, PTT, RMSSD, HRV LF/HF ratio, IBIS, and PPG entropy. The feature signals 255 can also include the mean or variance of a raw physiological signal 250 as a function of time or a time series that identifies the type of statistical distribution (e.g., uniform, Gaussian, Gamma) exhibited by a given raw physiological signal 250 as a function of time. As discussed above, in some embodiments feature extraction module 220 also selects the feature signals 255 that are relevant to the detection of a particular kind of physiological state of interest (target state).

At block 630, change point detection module 225 identifies change points 260 in the feature signals 255 (e.g., using a CPD algorithm). As discussed above and as those skilled in the art are aware, a CPD algorithm detects abrupt changes in the statistical distribution of a time series. An abrupt change can, for example, occur in the mean, variance, correlation length, or type of distribution (probability density function) itself. As those skilled in the art are aware, the analysis performed by a CPD algorithm is deterministic, but, in the embodiments disclosed herein, there is also a learned component, particularly in the algorithm's anomaly-detection thresholds (e.g., the amount by which the mean or variance of a physiological signal 250 must change to be considered an anomaly). As discussed above, the anomaly-detection thresholds can be adjusted over time based on a comparison of labeled anomalies (detected physiological states) with GT data 285 to improve the detection of specific physiological states (target states).

At block 640, change point detection module 225 identifies critical change points 265 in the feature signals 255 by applying a voting process to the change points 260 in the feature signals 255. As discussed above in connection with FIG. 4, the voting process can require that all the analyzed feature signals 255 include a change point 260 at a particular point in time (unanimous voting), or the voting process can be based on a majority of the analyzed feature signals 255 having a change point 260 at that point in time (majority voting), depending on the embodiment.

At block 650, segmentation module 230 partitions the feature signals 255 into segments 270 based on the critical change points 265. As discussed above, the critical change points 265 serve as delimiters for dividing the feature signals 255 into segments 270, as illustrated for the simple case of a single feature signal 255 (mean HR) in FIG. 3. In other words, the segments 270 are the portions of the feature signals 255 between critical change points 265.

At block 660, detection module 235 detects a predetermined physiological state of the human subject by applying clustering and probabilistic analysis to the segments 270. As discussed above, a probability-of-anomaly can be assigned to each segment 270 designated by segmentation module 230. In clustering the segments, detection module 235 employs density, distribution, or distance-based algorithms such as K-means clustering or a GMM to cluster the segments 270 (e.g., to label them as anomalous, the cluster with the greater distance being considered anomalous). This clustering and assignment of probability-of-anomaly ultimately identifies the segment 270 in which a hypothesized physiological state of interest (target state) most likely occurred. As also discussed above, this clustering process is performed (repeated) for each target state of interest. For example, the process might be performed for a symptomatic medical condition such as “motion sickness” and again for “irritation” and again for “stress.” The comparisons among segments 270 can also involve the individual subject's past physiological data (historical data 275 in FIG. 2). In comparing segments 270 to determine their similarities and differences, detection module 235 can employ techniques such as dynamic time warping (DTW). This is particularly useful for comparing segments of different durations.

At block 670, assistive action module 240, in response to detecting the predetermined physiological state, automatically takes an action to assist the human subject with regard to the predetermined physiological state. As discussed above, the action taken can, without limitation, include one or more of the following: (1) notifying the subject regarding the detected predetermined physiological state; (2) notifying the subject regarding a potentially unsafe driving condition stemming from the detected predetermined physiological state; (3) notifying the subject regarding a symptomatic illness (e.g., a heart condition or motion sickness) of the subject based on the detected predetermined physiological state; (4) advising the subject regarding mitigation of the detected physiological state (e.g., recommending that the subject take a break from driving or offering to turn on a particular kind of music in the vehicle to calm the driver or to increase the driver's alertness); (5) suggesting to the subject that the subject take an alternate route to a planned destination (e.g., a route that is less stressful for the driver); (6) communicating with a person associated with the subject (e.g., a friend or family member) or an AI system to obtain help for the subject; (7) assuming at least partial control over operation of the vehicle (route-planning, steering, braking, and/or acceleration) to mitigate a potentially unsafe driving condition stemming from the detected physiological state, where the vehicle includes an automated driving system or an Advanced Driver-Assistance System (ADAS); and (8) alerting other drivers on the roadway to potential danger stemming from the detected predetermined physiological state of the subject.

As also discussed above, in a non-vehicular embodiment in which the system 170 is implemented in a mobile device that is worn or carried by the subject, the action taken by assistive action module 240 can, without limitation, include one or more of the following: (1) notifying the subject regarding the detected predetermined physiological state; (2) notifying the subject regarding a symptomatic illness of the human subject (e.g., a heart condition) based on the detected physiological state; (3) advising the subject regarding mitigation of the detected physiological state (e.g., suggesting that the subject take a break or engage in a relaxing activity, suggesting that the subject seek medical attention, etc.); and (4) communicating with a person associated with the subject (e.g., a friend or family member) or an AI system to obtain help for the human subject.

As discussed above, method 600 can include evaluating the performance of the unsupervised machine-learning algorithm described herein by comparing a labeled physiological state (e.g., a particular emotion such as “annoyance”) with GT data 285 such as (1) a self-report questionnaire answered by the human subject, (2) event timestamps captured by one or more environmental sensors in the environment of the subject, or (3) both the questionnaire data and the event-timestamp data. As discussed above, in some embodiments, medical records can also be used as ground-truth data for existing medical disorders or illnesses. Based on that comparison, the system 170 can adjust one or more thresholds of the CPD algorithm to improve the accuracy of future anomaly detection and labeling.

It should also be mentioned that the system 170, in some embodiments, is updated frequently (possibly even daily). Each time the system 170 is updated, the self-report questionnaire 510 can again be employed as a source of GT data 285, as can event timestamps from environmental sensor data from sensors in the environment of the subject, whether in a vehicle 100 or another location. For a given individual subject, the need for incremental updates generally diminishes over time.

FIG. 1 will now be discussed in full detail as an example vehicle environment within which the systems and methods disclosed herein may be implemented. The vehicle 100 can include one or more processors 110. In one or more arrangements, the one or more processors 110 can be a main processor of the vehicle 100. For instance, the one or more processors 110 can be an electronic control unit (ECU). The vehicle 100 can include one or more data stores 115 for storing one or more types of data. The data store(s) 115 can include volatile and/or non-volatile memory. Examples of suitable data stores 115 include RAM, flash memory, ROM, PROM (Programmable Read-Only Memory), EPROM, EEPROM (Electrically Erasable Programmable Read-Only Memory), registers, magnetic disks, optical disks, hard drives, or any other suitable storage medium, or any combination thereof. The data store(s) 115 can be a component(s) of the one or more processors 110, or the data store(s) 115 can be operatively connected to the one or more processors 110 for use thereby. The term “operatively connected,” as used throughout this description, can include direct or indirect connections, including connections without direct physical contact.

In one or more arrangements, the one or more data stores 115 can include map data 116. The map data 116 can include maps of one or more geographic areas. In some instances, the map data 116 can include information or data on roads, traffic control devices, road markings, structures, features, and/or landmarks in the one or more geographic areas. In one or more arrangement, the map data 116 can include one or more terrain maps 117. The terrain map(s) 117 can include information about the ground, terrain, roads, surfaces, and/or other features of one or more geographic areas. In one or more arrangement, the map data 116 can include one or more static obstacle maps 118. The static obstacle map(s) 118 can include information about one or more static obstacles located within one or more geographic areas.

The one or more data stores 115 can include sensor data 119. In this context, “sensor data” means any information about the sensors that a vehicle is equipped with, including the capabilities and other information about such sensors. As will be explained below, the vehicle 100 can include the sensor system 120. The sensor data 119 can relate to one or more sensors of the sensor system 120. As an example, in one or more arrangements, the sensor data 119 can include information on one or more LIDAR sensors 124 of the sensor system 120. As discussed above, in some embodiments, vehicle 100 can receive sensor data from other connected vehicles, from devices associated with other road users (ORUs), or both.

As noted above, the vehicle 100 can include the sensor system 120. The sensor system 120 can include one or more sensors. “Sensor” means any device, component and/or system that can detect, and/or sense something. The one or more sensors can be configured to detect, and/or sense in real-time. As used herein, the term “real-time” means a level of processing responsiveness that a user or system senses as sufficiently immediate for a particular process or determination to be made, or that enables the processor to keep up with some external process.

In arrangements in which the sensor system 120 includes a plurality of sensors, the sensors can function independently from each other. Alternatively, two or more of the sensors can work in combination with each other. In such a case, the two or more sensors can form a sensor network. The sensor system 120 and/or the one or more sensors can be operatively connected to the one or more processors 110, the data store(s) 115, and/or another element of the vehicle 100 (including any of the elements shown in FIG. 1).

As discussed above, the sensor system 120 can include any suitable type of sensor. Various examples of different types of sensors will be described herein. However, it will be understood that the implementations are not limited to the particular sensors described. The sensor system 120 can include one or more vehicle sensors 121. The vehicle sensors 121 can detect, determine, and/or sense information about the vehicle 100 itself, including the operational status of various vehicle components and systems.

In one or more arrangements, the vehicle sensors 121 can be configured to detect, and/or sense position and/orientation changes of the vehicle 100, such as, for example, based on inertial acceleration. In one or more arrangements, the vehicle sensors 121 can include one or more accelerometers, one or more gyroscopes, an inertial measurement unit (IMU), a dead-reckoning system, a global navigation satellite system (GNSS), a navigation system 147, and/or other suitable sensors. The vehicle sensors 121 can be configured to detect, and/or sense one or more characteristics of the vehicle 100. In one or more arrangements, the vehicle sensors 121 can include a speedometer to determine a current speed of the vehicle 100.

Alternatively, or in addition, the sensor system 120 can include one or more environment sensors 122 configured to acquire, and/or sense driving environment data. “Driving environment data” includes any data or information about the external environment in which a vehicle is located or one or more portions thereof. For example, the one or more environment sensors 122 can be configured to detect, quantify, and/or sense obstacles in at least a portion of the external environment of the vehicle 100 and/or information/data about such obstacles. The one or more environment sensors 122 can be configured to detect, measure, quantify, and/or sense other things in at least a portion the external environment of the vehicle 100, such as, for example, nearby vehicles, lane markers, signs, traffic lights, traffic signs, lane lines, crosswalks, curbs proximate the vehicle 100, off-road objects, etc.

Various examples of sensors of the sensor system 120 will be described herein. The example sensors may be part of the one or more environment sensors 122 and/or the one or more vehicle sensors 121. Moreover, the sensor system 120 can include operator sensors that function to track or otherwise monitor aspects related to the driver/operator of the vehicle 100. However, it will be understood that the implementations are not limited to the particular sensors described. As an example, in one or more arrangements, the sensor system 120 can include one or more radar sensors 123, one or more LIDAR sensors 124, one or more sonar sensors 125, and/or one or more cameras 126.

As discussed above, a vehicle 100 can further include a communication system 130. The communication system 130 can include one or more components configured to facilitate communication between the vehicle 100 and one or more communication sources. Communication sources, as used herein, refers to people or devices with which the vehicle 100 can communicate with, such as external networks, computing devices, operator or occupants of the vehicle 100, or others. As part of the communication system 130, the vehicle 100 can include an input system 131. An “input system” includes any device, component, system, element or arrangement or groups thereof that enable information/data to be entered into a machine. In one or more examples, the input system 131 can receive an input from a vehicle occupant (e.g., a driver or a passenger). The vehicle 100 can include an output system 132. An “output system” includes any device, component, or arrangement or groups thereof that enable information/data to be presented to the one or more communication sources (e.g., a person, a vehicle passenger, etc.). The communication system 130 can further include specific elements which are part of or can interact with the input system 131 or the output system 132, such as one or more display device(s) 133, and one or more audio device(s) 134 (e.g., speakers and microphones).

The vehicle 100 can include one or more vehicle systems 140. Various examples of the one or more vehicle systems 140 are shown in FIG. 1. However, the vehicle 100 can include more, fewer, or different vehicle systems. It should be appreciated that although particular vehicle systems are separately defined, each or any of the systems or portions thereof may be otherwise combined or segregated via hardware and/or software within the vehicle 100. The vehicle 100 can include a propulsion system 141, a braking system 142, a steering system 143, throttle system 144, a transmission system 145, a signaling system 146, and/or a navigation system 147. Each of these systems can include one or more devices, components, and/or combinations thereof, now known or later developed.

The one or more processors 110 can be operatively connected to communicate with the various vehicle systems 140 and/or individual components thereof. For example, returning to FIG. 1, the one or more processors 110 can be in communication to send and/or receive information from the various vehicle systems 140 to control the movement, speed, maneuvering, heading, direction, etc. of the vehicle 100. The one or more processors 110 may control some or all of these vehicle systems 140.

The vehicle 100 can include one or more modules, at least some of which are described herein. The modules can be implemented as computer-readable program code that, when executed by a processor 110, implement one or more of the various processes described herein. The processor 110 can be a device, such as a CPU, which is capable of receiving and executing one or more threads of instructions for the purpose of performing a task. One or more of the modules can be a component of the one or more processors 110, or one or more of the modules can be executed on and/or distributed among other processing systems to which the one or more processors 110 is operatively connected. The modules can include instructions (e.g., program logic) executable by one or more processors 110. Alternatively, or in addition, one or more data store 115 may contain such instructions.

In one or more arrangements, one or more of the modules described herein can include artificial or computational intelligence elements, e.g., neural network, fuzzy logic or other machine learning algorithms. Further, in one or more arrangements, one or more of the modules can be distributed among a plurality of the modules described herein. In one or more arrangements, two or more of the modules described herein can be combined into a single module.

Detailed implementations are disclosed herein. However, it is to be understood that the disclosed implementations are intended only as examples. Therefore, specific structural and functional details disclosed herein are not to be interpreted as limiting, but merely as a basis for the claims and as a representative basis for teaching one skilled in the art to variously employ the aspects herein in virtually any appropriately detailed structure. Further, the terms and phrases used herein are not intended to be limiting but rather to provide an understandable description of possible implementations. Various implementations are shown in FIGS. 1-6, but the implementations are not limited to the illustrated structure or application.

The flowcharts and block diagrams in the figures illustrate the architecture, functionality, and operation of possible implementations of systems, methods, and computer program products according to various implementations. In this regard, each block in the flowcharts or block diagrams can represent a module, segment, or portion of code, which comprises one or more executable instructions for implementing the specified logical function(s). It should also be noted that, in some alternative implementations, the functions noted in the block can occur out of the order noted in the figures. For example, two blocks shown in succession can be executed substantially concurrently, or the blocks can sometimes be executed in the reverse order, depending upon the functionality involved.

The systems, components and/or methods described above can be realized in hardware or a combination of hardware and software and can be realized in a centralized fashion in one processing system or in a distributed fashion where different elements are spread across several interconnected processing systems. Any kind of processing system or other apparatus adapted for carrying out the methods described herein is suited. A typical combination of hardware and software can be a processing system with computer-usable program code that, when being loaded and executed, controls the processing system such that it carries out the methods described herein. The systems, components and/or methods also can be embedded in a computer-readable storage, such as a computer program product or other data programs storage device, readable by a machine, tangibly embodying a program of instructions executable by the machine to perform methods and methods described herein. These elements also can be embedded in an application product which comprises all the features enabling the implementation of the methods described herein and, which when loaded in a processing system, is able to carry out these methods.

Furthermore, arrangements described herein can take the form of a computer program product embodied in one or more computer-readable media having computer-readable program code embodied or embedded, such as stored thereon. Any combination of one or more computer-readable media can be utilized. The computer-readable medium can be a computer-readable signal medium or a computer-readable storage medium. The phrase “computer-readable storage medium” means a non-transitory storage medium. A computer-readable storage medium can be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk drive (HDD), a solid state drive (SSD), a RAM, a ROM, an EPROM or Flash memory, an optical fiber, a portable compact disc read-only memory (CD-ROM), a digital versatile disc (DVD), an optical storage device, a magnetic storage device, or any suitable combination of the foregoing. In the context of this document, a computer-readable storage medium can be any tangible medium that can contain, or store a program for use by, or in connection with, an instruction execution system, apparatus, or device.

Program code embodied on a computer-readable medium can be transmitted using any appropriate medium, including but not limited to wireless, wireline, optical fiber, cable, RF, etc., or any suitable combination of the foregoing. Computer program code for carrying out operations for aspects of the present arrangements can be written in any combination of one or more programming languages, including an object-oriented programming language such as Java™, Smalltalk, C++ or the like and conventional procedural programming languages, such as the “C” programming language or similar programming languages. The program code can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a LAN or a WAN, or the connection can be made to an external computer (for example, through the Internet using an Internet Service Provider).

In the description above, certain specific details are outlined in order to provide a thorough understanding of various implementations. However, one skilled in the art will understand that the invention may be practiced without these details. In other instances, well-known structures have not been shown or described in detail to avoid unnecessarily obscuring descriptions of the implementations. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one or more implementations” or “an implementation” means that a particular feature, structure or characteristic described in connection with the implementation is included in at least one or more implementations. Thus, the appearances of the phrases “in one or more implementations” or “in an implementation” in various places throughout this specification are not necessarily all referring to the same implementation. Furthermore, the particular features, structures, or characteristics may be combined in any suitable manner in one or more implementations. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings (such as “Background” and “Summary”) and sub-headings used herein are intended only for general organization of topics within the present disclosure and are not intended to limit the disclosure of the technology or any aspect thereof. The recitation of multiple implementations having stated features is not intended to exclude other implementations having additional features, or other implementations incorporating different combinations of the stated features. As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that an implementation can or may comprise certain elements or features does not exclude other implementations of the present technology that do not contain those elements or features.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one aspect, or various aspects means that a particular feature, structure, or characteristic described in connection with an implementation or particular system is included in at least one or more implementations or aspect. The appearances of the phrase “in one aspect” (or variations thereof) are not necessarily referring to the same aspect or implementation. It should also be understood that the various method steps discussed herein do not have to be carried out in the same order as depicted, and not each method step is required in each aspect or implementation.

Generally, “module,” as used herein, includes routines, programs, objects, components, data structures, and so on that perform particular tasks or implement particular data types. In further aspects, a memory generally stores the noted modules. The memory associated with a module may be a buffer or cache embedded within a processor, a RAM, a ROM, a flash memory, or another suitable electronic storage medium. In still further aspects, a module as envisioned by the present disclosure is implemented as an application-specific integrated circuit (ASIC), a hardware component of a system on a chip (SoC), as a programmable logic array (PLA), or as another suitable hardware component that is embedded with a defined configuration set (e.g., instructions) for performing the disclosed functions.

The terms “a” and “an,” as used herein, are defined as one as or more than one. The term “plurality,” as used herein, is defined as two or more than two. The term “another,” as used herein, is defined as at least a second or more. The terms “including” and/or “having,” as used herein, are defined as including (i.e., open language). The phrase “at least one of . . . and . . . ” as used herein refers to and encompasses any and all possible combinations of one or more of the associated listed items. As an example, the phrase “at least one of A, B and C” includes A only, B only, C only, or any combination thereof (e.g., AB, AC, BC or ABC).

The preceding description of the implementations has been provided for purposes of illustration and description. It is not intended to be exhaustive or to limit the disclosure. Individual elements or features of a particular implementation are generally not limited to that particular implementation, but, where applicable, are interchangeable and can be used in a selected implementation, even if not specifically shown or described. The same may also be varied in many ways. Such variations should not be regarded as a departure from the disclosure, and all such modifications are intended to be included within the scope of the disclosure.

While the preceding is directed to implementations of the disclosed devices, systems, and methods, other and further implementations of the disclosed devices, systems, and methods can be devised without departing from the basic scope thereof. The scope thereof is determined by the claims that follow.

What is claimed is:

1. A system for identifying physiological states of human subjects, the system comprising:
a processor; and
a memory storing machine-readable instructions that, when executed by the processor, cause the processor to:
receive, from one or more sensors, physiological signals of a human;
process the physiological signals to extract feature signals as time series;
identify change points in the feature signals;
identify critical change points in the feature signals by applying a voting process to the change points in the feature signals;
partition the feature signals into segments based on the critical change points;
detect a predetermined physiological state of the human by applying clustering and probabilistic analysis to the segments; and
take automatically, in response to detecting the predetermined physiological state, an action to assist the human.

2. The system of claim 1, wherein the machine-readable instructions include further instructions that, when executed by the processor, cause the processor to compare the detected predetermined physiological state with one of a self-report questionnaire of the human and event timestamps captured by one or more environmental sensors in an environment of the human to adjust one or more thresholds of a change point detection algorithm.

3. The system of claim 1, wherein the predetermined physiological state is one of a symptomatic illness, an emotion, stress, and drowsiness.

4. The system of claim 3, wherein the symptomatic illness is one of a heart condition and motion sickness.

5. The system of claim 1, wherein the human is a driver of a vehicle equipped with the system.

6. The system of claim 5, wherein the action includes one or more of:
notifying the human regarding the detected predetermined physiological state;
notifying the human regarding a potentially unsafe driving condition stemming from the detected predetermined physiological state;
notifying the human regarding a symptomatic illness of the human based on the detected predetermined physiological state;
advising the human regarding mitigation of the detected physiological state;
suggesting to the human that the human take an alternate route to a planned destination;
communicating with one of a person associated with the human and an artificial-intelligence (AI) system to obtain help for the human;
assuming at least partial control over operation of the vehicle to mitigate a potentially unsafe driving condition stemming from the detected predetermined physiological state; and
alerting other drivers on the roadway to potential danger stemming from the detected predetermined physiological state.

7. The system of claim 1, wherein the system is embodied in a mobile device that is one of carried and worn by the human and the one or more sensors are one of integral with the mobile device and separate from the mobile device.

8. The system of claim 7, wherein the action includes one or more of:
notifying the human regarding the detected predetermined physiological state;
notifying the human regarding a symptomatic illness of the human based on the detected predetermined physiological state;
advising the human regarding mitigation of the detected physiological state; and
communicating with one of a person associated with the human and an artificial-intelligence (AI) system to obtain help for the human.

9. A non-transitory computer-readable medium for identifying physiological states of human subjects and storing instructions that, when executed by a processor, cause the processor to:
receive, from one or more sensors, physiological signals of a human;
process the physiological signals to extract feature signals as time series.
identify change points in the feature signals;
identify critical change points in the feature signals by applying a voting process to the change points in the feature signals;
partition the feature signals into segments based on the critical change points;
detect a predetermined physiological state of the human by applying clustering and probabilistic analysis to the segments; and
take automatically, in response to detecting the predetermined physiological state, an action to assist the human.

10. The non-transitory computer-readable medium of claim 9, wherein the instructions include further instructions that, when executed by the processor, cause the processor to compare the detected predetermined physiological state with one of a self-report questionnaire of the human and event timestamps captured by one or more environmental sensors in an environment of the human to adjust one or more thresholds of a change point detection algorithm.

11. The non-transitory computer-readable medium of claim 9, wherein the human is a driver of a vehicle equipped with a system that includes the non-transitory computer-readable medium.

12. The non-transitory computer-readable medium of claim 11, wherein the action includes one or more of:
notifying the human regarding the detected predetermined physiological state;
notifying the human regarding a potentially unsafe driving condition stemming from the detected predetermined physiological state;
notifying the human regarding a symptomatic illness of the human based on the detected predetermined physiological state;
advising the human regarding mitigation of the detected physiological state;
suggesting to the human that the human take an alternate route to a planned destination;
communicating with one of a person associated with the human and an artificial-intelligence (AI) system to obtain help for the human;
assuming at least partial control over operation of the vehicle to mitigate a potentially unsafe driving condition stemming from the detected predetermined physiological state; and
alerting other drivers on the roadway to potential danger stemming from the detected predetermined physiological state.

13. The non-transitory computer-readable medium of claim 9, wherein the non-transitory computer-readable medium is part of a mobile device that is one of carried and worn by the human and the one or more sensors are one of integral with the mobile device and separate from the mobile device.

14. The non-transitory computer-readable medium of claim 13, wherein the action includes one or more of:
notifying the human regarding the detected predetermined physiological state;
notifying the human regarding a symptomatic illness of the human based on the detected predetermined physiological state;
advising the human regarding mitigation of the detected physiological state; and
communicating with one of a person associated with the human and an artificial-intelligence (AI) system to obtain help for the human.

15. A method, comprising:
receiving, from one or more sensors, physiological signals of a human and processing the physiological signals to extract feature signals as time series;
identifying change points in the feature signals;
identifying critical change points in the feature signals by applying a voting process to the change points in the feature signals and partitioning the feature signals into segments based on the critical change points;
detecting a predetermined physiological state of the human by applying clustering and probabilistic analysis to the segments; and
taking automatically, in response to detecting the predetermined physiological state, an action to assist the human.

16. The method of claim 15, further comprising:
comparing the detected predetermined physiological state with one of a self-report questionnaire of the human and event timestamps captured by one or more environmental sensors in an environment of the human to adjust one or more thresholds of a change point detection algorithm.

17. The method of claim 15, wherein the human is a driver of a vehicle equipped with a system in which the method is implemented.

18. The method of claim 17, wherein the action includes one or more of:
notifying the human regarding the detected predetermined physiological state;
notifying the human regarding a potentially unsafe driving condition stemming from the detected predetermined physiological state;
notifying the human regarding a symptomatic illness of the human based on the detected predetermined physiological state;
advising the human regarding mitigation of the detected physiological state;
suggesting to the human that the human take an alternate route to a planned destination;
communicating with one of a person associated with the human and an artificial-intelligence (AI) system to obtain help for the human;
assuming at least partial control over operation of the vehicle to mitigate a potentially unsafe driving condition stemming from the detected predetermined physiological state; and
alerting other drivers on the roadway to potential danger stemming from the detected predetermined physiological state.

19. The method of claim 15, wherein the method is implemented in a mobile device that is one of carried and worn by the human and the one or more sensors are one of integral with the mobile device and separate from the mobile device.

20. The method of claim 19, wherein the action includes one or more of:
notifying the human regarding the detected predetermined physiological state;
notifying the human regarding a symptomatic illness of the human based on the detected predetermined physiological state;
advising the human regarding mitigation of the detected physiological state; and
communicating with one of a person associated with the human and an artificial-intelligence (AI) system to obtain help for the human.
